(19) United States
(12) Patent Application Publication
(10) Pub. No.: US 2010/0047366 A1
(43) Pub. Date: Feb. 25, 2010

(54) ARTICLES WITH ANTIBACTERIAL AND ANTIFUNGAL ACTIVITY

(75) Inventors: Thierry CHARBONNEAUX, Lentate Sul Seveso (IT); Sandrine Rochat, Villeurbanne (FR)

Correspondence Address: BUCHANAN, INGERSOLL & ROONEY PC, POST OFFICE BOX 1404, ALEXANDRIA, VA, 22313-1404, US

(73) Assignee: RHODIANYL, Paris La Defense Cedex (FR)

(21) Appl. No.: 12/611,419

(22) Filed: Nov. 3, 2009

Related U.S. Application Data
(62) Division of application No. 10/534,209, filed on Apr. 18, 2006.

(30) Foreign Application Priority Data
Nov. 8, 2002 (FR) 02/14007

(51) Int. Cl. A61K 33/30; A61P 31/04; A61P 31/10

(52) U.S. Cl. 424/641

(57) ABSTRACT

The present invention relates to articles with antibacterial and antifungal activity, comprising zinc sulphide. The yarns, fibres, filaments and articles according to the present invention may be used especially in the manufacture of any product liable to be placed in contact with bacteria and/or fungi, for instance clothing, rugs, curtains, bedclothes and medical textile materials. The present invention also relates to the use of zinc sulphide for manufacturing yarns, fibres, filaments and/or articles with antibacterial and antifungal properties.

The present invention relates to articles with antibacterial and antifungal activity, comprising zinc sulphide. The yarns, fibres, filaments and articles according to the present invention may be used especially in the manufacture of any product liable to be placed in contact with bacteria and/or fungi, for instance clothing, rugs, curtains, bedclothes and medical textile materials. The present invention also relates to the use of zinc sulphide for manufacturing yarns, fibres, filaments and/or articles with antibacterial and antifungal properties.

In numerous applications such as the textile field, it is sought to limit the development of bacteria and fungi, for the purpose of preventing diseases in man and to avoid unpleasant odours. In the medical sector, for example, it is also of great importance to limit the growth of bacteria and fungi on work tools, construction materials and clothing.

Many agents with biocidal properties have been known for a very long time and are used in various applications. Among these agents, components based on metals such as silver, copper or zinc, based on quaternary ammonium, or organic-based components, for instance triclosan, are the most commonly known.

In order to give textile surfaces biocidal properties, numerous finishings containing bioactive compounds have been developed. However, these finishings always have limited fastness and their effects disappear after one or more washes. It is thus more advantageous in many cases to introduce the active principle directly into the article that needs to have a bioactive property.

Many commercial antibacterial and antifungal agents are known. However, these agents cannot be introduced into polymer matrices, since they do not withstand the forming temperatures of these polymer matrices, and may be converted at these temperatures or may interact with the matrix.

Novel inexpensive antibacterial and antifungal agents that are easy to use in articles based on a polymer matrix are still being sought.

The Applicant has demonstrated that yarns, fibres, filaments and/or articles, such as films, comprising zinc sulphide (ZnS) in their polymer matrix have excellent antibacterial and antifungal properties. These antimicrobial properties are imparted by adding zinc sulphide as a mineral filler to the polymer matrix.

The zinc sulphide disperses readily in the polymer matrix, thus allowing a uniform distribution of this compound in the yarns, fibres, filaments and/or articles. Zinc sulphide does not aggregate in the polymer matrix, unlike many metal-based particles known in the prior art as antimicrobial agents.

By diffusion and migration, the active principle, in the form of zinc sulphide and/or of zinc, is released at the surface of the yarns, fibres, filaments and/or articles and comes into contact with the environment comprising the bacteria and fungi, thus allowing longer-lasting antibacterial and antifungal activity. On washing the yarns, fibres and/or filaments, a small amount of the active principle is removed from the surface. However, the diffusion of the active principle in the polymer matrix from the core to the surface of the yarns, fibres, filaments and/or articles allows the antibacterial and antifungal activity to be kept constant. This activity is thus preserved for a very long time, despite washing the yarns, fibres, filaments and/or articles.

Zinc sulphide also has the advantage of withstanding the forming temperatures of the thermoplastic matrix. Zinc sulphide is therefore not modified or altered at these temperatures.

Furthermore, zinc sulphide is inert and does not react with the polymer matrix, thus causing no problems of degradation, coloration or yellowing of the yarns, fibres, filaments and/or articles, unlike the antimicrobial agents of the prior art, for instance zinc oxide (ZnO) or silver (Ag). Furthermore, the yarns, fibres, filaments and/or articles comprising zinc sulphide are not abrasive.

Zinc sulphide also makes it possible to satisfy the desired properties in terms of cost, ease of use and of introduction into polymer matrices, such as thermoplastic matrices. Zinc sulphide also has the advantage of being a good delustrant.

The term “antibacterial” means the action intended to limit, reduce or eliminate the bacteria present in an environment. The term “bacteria” means eubacteria and archeobacteria. Eubacteria include fermicutes, gracilicutes and ternicutes. Gracilicutes include Gram-negative bacteria such as the Enterobacteriaceae, for instance Klebsiella (such as Klebsiella pneumoniae) and Escherichia (such as Escherichia coli). The fermicutes include Gram-positive bacteria, such as Micrococcaceae, for instance Staphylococci (such as Staphylococcus aureus) and endospore-forming rods including the bacilli (Bacillaceae), for instance Bacillus circulans. All these references are mentioned in Bergey's Manual of Systematic Bacteriology, Williams & Wilkens, 1st ed., Vol. 1-4, (1984).

The term “antifungal” means the action intended to limit, reduce or eliminate the fungi (mycetes) present in an environment. The term Myceteae includes Amastigomycota, for instance Deuteromycotina which includes the Deuteromycetes. The Deuteromycetes include Aspergillis (Aspergillus niger) and Candida (Candida albicans).

The term “environment” means any medium comprising at least bacteria and/or fungi. The environment may be a liquid or a gas, preferably air. The term “reduce” means to decrease the amount of bacteria and/or fungi present in the environment, compared with the amount present in the environment before the introduction of yarns comprising zinc sulphide. The term “reduce” also means to reduce the rate of growth of the new bacteria and/or fungi over time and in the environment. The term “reduce” also means to reduce the rate of reproduction of the bacteria and/or fungi. The term “eliminate” means to eliminate from the environment the majority of the bacteria and/or fungi, i.e. to kill the bacteria and/or fungi present in the environment or to render them inactive. The term “eliminate” also means to prevent the growth of new bacteria and/or fungi.

The present invention also relates to the use of zinc sulphide in a polymer matrix for the manufacture of yarns, fibres, filaments and/or articles with antibacterial and antifungal properties. Zinc sulphide acts therein as an antibacterial and antifungal agent.

A first subject of the present invention is yarns, fibres and/or filaments with antibacterial and antifungal properties, comprising a polymer matrix and zinc sulphide.

The presence of zinc sulphide in a polymer matrix may be determined by various methods that are well known to those skilled in the art, such as a direct qualitative analysis of the elements zinc and sulphur by X-ray fluorescence spectrometry; optionally followed by quantitative elemental assay of the element zinc after sulphonitric mineralization by atomic spectrometry, so as to deduce therefrom the amount of zinc sulphide. It is also possible to quantitatively determine the element sulphur by microanalysis and/or to dissolve the polymer matrix in a solvent, filter off the additive and perform an analysis by X-ray diffraction.

The weight proportion of zinc sulphide relative to the total weight of the composition intended to form the yarns, fibres and/or filaments may be between 0.01% and 10%, preferably between 0.1% and 7%, even more preferably between 0.2% and 5% and particularly between 0.3% and 3%. The amount of zinc sulphide in the yarns, fibres and/or filaments may vary according to different criteria, such as the level of delustring, the formulation, the type of polymer, the introduction method, the application method, the nature of the harmful organisms and the environment.

As examples of polymers of which the polymer matrix is composed, mention may be made of: polylactones such as poly(pivalolactone), poly(caprolactone) and polymers of the same family; polyurethanes obtained by reaction between diisocyanates, for instance 1,5-naphthalene diisocyanate; p-phenylene diisocyanate, m-phenylene diisocyanate, 2,4-toluene diisocyanate, 4,4′diphenylmethane diisocyanate, 3,3′-dimethyl-4,4′-diphenylmethane diisocyanate, 3,3′-dimethyl-4,4′-biphenyl diisocyanate, 4,4′-diphenylisopropylidene diisocyanate, 3,3′-dimethyl-4,4′-diphenyl diisocyanate, 3,3′-dimethyl-4,4′-diphenylmethane diisocyanate, 3,3′-dimethoxy-4,4′-biphenyl diisocyanate, dianisidine diisocyanate, toluidine diisocyanate, hexamethylene diisocyanate, 4,4′-diisocyanatodiphenylmethane and compounds of the same family and linear long-chain diols, for instance poly(tetramethylene adipate), poly(ethylene adipate), poly(1,4-butylene adipate), poly(ethylene succinate), poly(2,3-butylene succinate), polyetherdiols and compounds of the same family; polycarbonates, for instance poly[methanebis(4-phenyl)carbonate], poly[1,1-etherbis(4-phenyl)carbonate], poly[diphenyl-methanebis(4-phenyl)carbonate], poly[1,1-cyclohexane-bis(4-phenyl)carbonate] and polymers of the same family; polysulphones; polyethers; polyketones; polyamides, for instance poly(4-aminobutyric acid), poly(hexamethyleneadipamide), poly(ε-caprolactam), poly(6-aminohexanoic acid), poly(m-xylyleneadipamide), poly(p-xylylenesebacamide), poly(2,2,2-trimethylhexamethyleneterephthalamide), poly(meta-phenyleneisophthalamide), poly(p-phenyleneterephthalamide) and polymers of the same family; polyesters, for instance poly(ethylene azelate), poly(ethylene 1,5-naphthalate), poly(1,4-cyclohexanedimethylene terephthalate), poly(ethylene oxybenzoate), poly(para-hydroxybenzoate), poly(1,4-cyclohexylidenedimethylene terephthalate), poly(1,4-cyclohexylidenedimethylene terephthalate), polyethylene terephthalate, polybutylene terephthalate and polymers of the same family; poly(arylene oxides), for instance poly(2,6-dimethyl-1,4-phenylene oxide), poly(2,6-diphenyl-1,4-phenylene oxide) and polymers of the same family; poly(arylene sulphides), for instance poly(phenylene sulphide) and polymers of the same family; polyetherimides; vinyl polymers and copolymers thereof, for instance polyvinyl acetate, polyvinyl alcohol, polyvinyl chloride; polyvinyl butyral, polyvinylidene chloride, ethylene/vinyl acetate copolymers, and polymers of the same family; acrylic polymers, polyacrylates and copolymers thereof, for instance polyethyl acrylate, poly(n-butyl acrylate), polymethyl methacrylate, polyethyl methacrylate, poly(n-butyl methacrylate), poly(n-propyl methacrylate), polyacrylamide, polyacrylonitrile, poly(acrylic acid), ethylene/acrylic acid copolymers, ethylene/vinyl alcohol copolymers, acrylonitrile copolymers, methyl methacrylate/styrene copolymers, ethylene/ethyl acrylate copolymers, methacrylate/butadiene/styrene copolymers, ABS, and polymers of the same family; polyolefins, for instance low-density poly(ethylene), poly(propylene), low-density chlorinated poly(ethylene), poly(4-methyl-1-pentene), poly(ethylene), poly(styrene), and polymers of the same family; ionomers; poly(epichlorohydrins); poly(urethanes) such as the products of polymerization of diols, for instance glycerol, trimethylolpropane, 1,2,6-hexanetriol, sorbitol, pentaerythritol, polyetherpolyols, polyesterpolyols and compounds of the same family, with polyisocyanates, for instance 2,4-tolylene diisocyanate, 2,6-tolylene diisocyanate, 4,4′-diphenylmethane diisocyanate, 1,6-hexamethylene diisocyanate, 4,4′-dicyclohexylmethane diisocyanate and compounds of the same family; and polysulphones, such as the products of reaction between a sodium salt of 2,2-bis(4-hydroxyphenyl)propane and 4,4′-dichloro-diphenyl sulphone; furan resins, for instance poly(furan); cellulose-ester plastics, for instance cellulose acetate, cellulose acetate-butyrate, cellulose propionate and polymers of the same family; silicones, for instance poly(dimethylsiloxane), poly(dimethylsiloxane-co-phenylmethylsiloxane), and polymers of the same family; blends of at least two of the above polymers.

As other polymer matrices, mention may also be made, for example, of viscose, cellulose and cellulose acetate; polyamideimides or polyimides; latices, such as acrylic and urethane latices.

The polymer matrix of the invention may also be of the type of polymers used in adhesives, for instance copolymers of plastisol vinyl acetates, acrylic latices, urethane latices and plastisol PVCs.

The polymer matrix is preferably a thermoplastic matrix.

Preferably, the yarns, fibres and/or filaments of the present invention comprise a thermoplastic matrix composed of a thermoplastic polymer chosen from the group comprising polyamides; polyesters such as polyethylene terephthalate (PET), polybutylene terephthalate (PBT), polytrimethylene terephthalate (PTT); polyolefins such as polypropylene, polyethylene; polyvinylidene chloride (PVC), and copolymers and blends thereof.

Preferably, the thermoplastic matrix comprises at least one polyamide chosen from the group comprising: polyamide 6, polyamide 6,6, polyamide 11, polyamide 12, polyamide 4, polyamides 4-6, 6-10, 6-12, 6-36 and 12-12, and copolymers and blends thereof, such as a blend of polyamide 6 and 6,6. It is also possible to use different types of aromatic polyamide.

According to one particular variant of the invention, the thermoplastic matrix is a polymer comprising starburst or H-shaped macromolecular chains and, where appropriate, linear macromolecular chains. Polymers comprising such starburst or H-shaped macromolecular chains are described, for example, in documents FR 2 743 077, FR 2 779 730, U.S. Pat. No. 5,959,069, EP 0 632 703, EP 0 682 057 and EP 0 832 149.

The thermoplastic matrix of the invention may also be a polymer of random arborescent type, preferably a copolyamide with a random arborescent structure. These copolyamides of random arborescent structure and the process for obtaining them are described especially in document WO 99/03909. The thermoplastic matrix of the invention may also be a composition comprising a linear thermoplastic polymer and a starburst, H-shaped and/or arborescent thermoplastic polymer as described above. The thermoplastic matrix of the invention may also comprise a hyperbranched copolyamide of the type described in document WO 00/68298. The thermoplastic matrix of the invention may also comprise any combination of hyperbranched copolyamide, arborescent, H-shaped or starburst thermoplastic polymer described above.

The zinc sulphide may be in the form of particles. The zinc sulphide particles may have a diameter of less than or equal to 5 μm, preferably less than or equal to 1 μm, more preferably between 0.1 and 0.5 μm, and particularly a diameter of about 0.3 μm.

Preferably, the yarns, fibres and/or filaments of the present invention exclusively comprise zinc sulphide as antibacterial and antifungal agent. However, the zinc sulphide may be used in combination with at least one other antimicrobial agent, for instance silver, silver oxide, a silver halide, copper (I) oxide, copper (II) oxide, copper sulphide, zinc oxide and zinc silicate, a person skilled in the art being capable of selecting the nature and proportion of antimicrobial agent according to the use, the application method, the nature of the harmful organisms, the nature of the fibres, yarns, filaments and/or articles, and the environment.

The zinc sulphide introduced into the polymer matrix may be in the form of particles that are neither coated nor encapsulated. However, these particles may also be coated and/or encapsulated. The zinc sulphide particles may be coated and/or encapsulated with at least one mineral and/or organic compound. It is possible to use zinc sulphide particles not comprising a mineral coating.

The yarns, fibres, filaments and/or articles of the present invention may also contain any other additives that may be used, for example reinforcing fillers, flame retardants, UV stabilizers, heat stabilizers, pigments and delustrants such as titanium dioxide.

The present invention also relates to a process for manufacturing yarns, fibres and/or filaments with antibacterial and antifungal properties, which consists in spinning a composition comprising a polymer matrix, preferably a thermoplastic composition, and zinc sulphide.

The mixture of zinc sulphide and of the polymer matrix may be prepared in various ways that are well known to those skilled in the art. The compositions comprising a polymer matrix and zinc sulphide according to the invention are preferably prepared by introducing zinc sulphide into the polymer melt in a mixing device, for example upstream of a spinning device. They may also be prepared by introducing zinc sulphide into a polymer solution, for example upstream of a wet spinning device. The compositions may also be prepared by introducing zinc sulphide before the polymerization (with the raw materials) and/or during the polymerization of the polymer matrix, which is preferably thermoplastic. A concentrated composition (masterbatch) based on a polymer matrix comprising zinc sulphide may be added to the polymer matrix.

It is especially possible to use the following process comprising at least the steps:
a) placing the polymer matrix, optionally in melt form, in contact with zinc sulphide and/or a concentrated composition based on polymer matrix comprising zinc sulphide; and
b) spinning the mixture obtained in step a) so as to obtain yarns, fibres and/or filaments.

The compositions may be shaped into yarns, fibres and/or filaments directly after the polymerization, without intermediate solidification and remelting steps. They may also be shaped into granules, intended to undergo remelting for subsequent final shaping, for example for the manufacture of moulded articles or for the manufacture of yarns, fibres or filaments.

Any melt-spinning process may be used.

For the manufacture of multifilament yarns, mention may be made of the processes of integrated or non-integrated spinning or spin-drawing or spin-drawing-texturing, irrespective of the spinning speed. Yarns may be produced by high-speed spinning, at a spinning speed of greater than 3 500 m/min. Such processes are often denoted by the following terms: POY (partially oriented yarn), FOY (fully oriented yarn), ISD (integrated spin-drawing).

For the manufacture of fibres, the filaments may be combined, for example, in the form of roving or a lap, directly after spinning or take-up, drawn, textured or crinkled and chopped. The fibres obtained may be used for the manufacture of nonwovens or fibre yarns. The compositions may also be used for the manufacture of flock.

It is also possible to produce bicomponent yarns, fibres and/or filaments, certain parts of which comprise zinc sulphide.

The yarns, fibres and/or filaments of the invention may undergo various treatments, for example drawing in a continuous step or take-up drawing, deposition of size, oiling, structuring, texturing, crimping, drawing, fixing or relaxing heat treatment, throwing, twisting and/or dyeing. For dyeing, mention is made in particular of the processes of vat dyeing or jet dyeing. The preferred dyes are acid dyes, metalliferous dyes or non-metalliferous dyes.

The present invention also relates to an article with antibacterial and antifungal properties obtained at least from yarns, fibres and/or filaments as defined above. These articles may be textile surfaces or fabrics, such as woven, knitted or nonwoven surfaces or rugs. Specifically, the yarns, fibres, filaments, articles and/or composite articles may be used in the manufacture of any article liable to come into contact with bacteria and/or fungi, for instance carpets, rugs, furniture coverings, surface coverings, sofas, curtains, bedclothes, mattresses and pillows, clothing and medical textile materials.

Such articles may be obtained especially from yarns, fibres and/or filaments of a single type; or, on the contrary, from a blend of yarns, fibres and/or filaments of different types. The article at least partially comprises yarns, fibres and/or filaments according to the invention. For yarns, fibres or filaments of a given type, for example yarns, fibres or filaments not containing zinc sulphide, yarns, fibres or filaments of different nature may be used in the article of the invention. The present invention also relates to composite articles with antibacterial and antifungal properties, comprising at least one article according to the invention. The composite articles are multi-component articles. These components may be, for example, short fibres, supports, articles obtained from yarns, fibres or filaments, such as nonwoven articles. In the context of the invention, at least one of the components of the composite textile article comprises zinc sulphide.

The present invention also relates to articles obtained by forming a composition based on a polymer matrix comprising at least zinc sulphide. These articles may be obtained especially by a process chosen from the group comprising an extrusion process, such as the extrusion of sheets and films, a moulding process, such as compression-moulding, and an injection process, such as injection-moulding. Films may thus be obtained by the processes mentioned above using a flat die. Preferably, the thermoplastic matrix is composed of polyamide, polyester or polyolefin. The films obtained may undergo one or more treatment steps, such as one-dimensional or two-dimensional drawing, a stabilizing heat treatment, an antistatic treatment or a sizing operation.

### EXAMPLE 1

### Preparation of the Samples

A standard polyamide 66 with a relative viscosity of 2.6 (measured at 1 g/100 mL in 96% sulphuric acid at 25° C.) is dried conventionally to obtain a residual humidity of 0.09%. It is then reduced to powder and mixed with 2% ZnS powder (Sachtolith HD-S from Sachtleben—Germany). The resulting mixture is melted in an extruder and spun in a die with 10 die holes, thus creating 10 filaments, which are cooled by blowing with air (20° C., 66% relative humidity). The filaments are then combined and oiled with a standard 8.6% emulsion, and then wound onto a tube at 4 200 m/min. The resulting partially oriented yarn (POY), having an overall yarn count of 42 dtex, is then knitted on a conventional machine to obtain an article (a sock). This article is then subjected to a dyeing cycle under the following conditions:
desizing at 60° C. for 20 minutes with 1 g/L of an anionic detergent (Invatex CRA from Ciba) and 1 g/L of sodium carbonate;
open vat dyeing (volume: 7 L), with heating of 1.6° C./min, followed by maintenance at 98° C. for 45 minutes. The formula contains 1% Nylosan Blue NBLN (Clariant), 1% Sandogen NH (equalizer from Clariant), 1 g/L of Sandacid VA (acid donor from Clariant) and 0.5 g/L of sodium acetate.

An article obtained without addition of ZnS was also manufactured under the same conditions, as a control sample for the antibacterial and antifungal tests.

### EXAMPLE 2

### Antibacterial Test

The antibacterial activity is measured according to Standard JIS L 1902: 1998, following the particular procedure of the Hygiene and Biotechnology Laboratory of the Hohenstein Institut (Germany):
the bacteria Staphylococcus aureus ATCC 6538P and Klebsiella pneumoniae DSM 789, initially maintained in dry form and frozen, are used. The cultures are inoculated onto a nutrient base (LAB8, LabM), and incubated at 37° C. for 48 hours. The bacteria are then transferred into 250 ml conical flasks, onto a nutrient base (LAB14, LabM) and incubated at 37° C. for 18 hours. The culture is diluted to 1/200 with isotonic saline solution (0.85 weight % NaCl+0.05% Tween 80) such that the suspension comprises about 10⁵ bacteria per ml.
The tests are performed on 18 mm×18 mm knitted surfaces. As many surfaces as required to exactly absorb 0.2 ml of suspension are used.

The test samples are a control sample and a sample according to the invention.

The knitted surfaces are placed in 30 ml bottles. Six bottles are prepared comprising control samples, and three bottles for the test sample according to the invention. The bottles are covered with a film and sterilized at 121° C. for 15 minutes.

The bacteria are inoculated onto the samples included in the bottles with the 0.2 ml of the bacterial suspension, taking care not to place the suspension in contact with the walls of the bottle. Immediately after the inoculation, 20 ml of an isotonic Tween 80 solution (0.2% by weight) are added to three of the bottles containing the control sample, they are closed with a sterile stopper and are shaken for 30 seconds. The number of bacteria is then counted.

The other bottles are placed in a desiccator and the bacteria are left to incubate for 18 hours at 37+ C. After incubating, the number of bacteria is counted, in the same way as the number of bacteria at time zero.

The following amounts are determined in particular:
A=average number of active bacteria immediately after the inoculation on the control sample
B=average number of active bacteria after 18 hours of incubation on the control sample
C=average number of active bacteria after 18 hours of incubation on the sample according to the invention (with ZnS)
F=growth factor=Log(B)−Log(A). The test is considered as valid if F>0±0.5
S=specific activity=Log(B)−Log(C) cfu (colony-forming unit)

The results are collated in Tables 1 and 2 for the Gram-positive and Gram-negative bacteria.

TABLE 1
Staphylococcus aureus (Gram-positive): Strain ATCC 6538P

| | | | | | Mean | |
|---|---|---|---|---|---|---|
| | Sample | Sample | Sample | Mean | (Log | |
| | 1 (cfu) | 2 (cfu) | 3 (cfu) | (cfu) | cfu) | |
| Control | 4.50 × 10⁵ | 3.60 × 10⁵ | 4.50 × 10⁵ | 4.20 × 10⁵ | 5.62 | |
| 0 h | | | | | | |
| Control | 4.64 × 10⁵ | 8.39 × 10⁵ | 8.70 × 10⁵ | 7.25 × 10⁵ | 5.86 | F = |
| 18 h | | | | | | 0.24 |
| Test | <20 | 4.07 × 10² | <20 | 1.36 × 10² | 2.13 | S = |
| 18 h | | | | | | 3.87 |

TABLE 2
Klebsiella pneumoniae (Gram-negative): Strain DSM 789

| | | | | | Mean | |
|---|---|---|---|---|---|---|
| | Sample | Sample | Sample | Mean | (Log | |
| | 1 (cfu) | 2 (cfu) | 3 (cfu) | (cfu) | cfu) | |
| Control | 2.15 × 10⁵ | 6.70 × 10⁵ | 7.40 × 10⁵ | 5.42 × 10⁵ | 5.73 | |
| 0 h | | | | | | |
| Control | 3.58 × 10⁷ | 4.00 × 10⁷ | 4.28 × 10⁷ | 3.95 × 10⁷ | 7.60 | F = |
| 18 h | | | | | | 1.86 |
| Test | 2.28 × 10⁷ | 2.71 × 10⁷ | 2.76 × 10⁷ | 2.58 × 10⁷ | 7.41 | S = |
| 18 h | | | | | | 0.18 |

Thus, it is seen that the articles obtained from yarns comprising ZnS show a high antibacterial activity on the Gram-positive and Gram-negative bacteria.

### EXAMPLE 3

### Remanance of the Antibacterial Activity After Washing

The two samples (control sample and sample according to the invention) prepared above are washed 30 times each according to standard EN 26330—protocol 5A: the washing temperature is 40° C., the detergent used is free of optical brightener, and the machine used is a standard domestic machine. The samples are dried at room temperature.

The antibacterial activity is then re-measured according to the same methodology as above. The results are collated in Tables 3 and 4.

TABLE 3
Staphylococcus aureus (Gram-positive): Strain ATCC 6538P

| | | | | | Mean | |
|---|---|---|---|---|---|---|
| | Sample | Sample | Sample | Mean | (Log | |
| | 1 (cfu) | 2 (cfu) | 3 (cfu) | (cfu) | cfu) | |
| Control | 3.40 × 10⁵ | 3.10 × 10⁵ | 3.80 × 10⁵ | 3.43 × 10⁵ | 5.54 | |
| 0 h | | | | | | |
| Control | 8.10 × 10² | <20 | <20 | 2.71 × 10² | 2.43 | F = |
| 18 h | | | | | | −3.1 |
| Test | <20 | <20 | <20 | <20 | 0.01 | S = |
| 18 h | | | | | | 2.42 |

TABLE 4
Klebsiella pneumoniae (Gram-negative): Strain DSM 789

| | | | | | Mean | |
|---|---|---|---|---|---|---|
| | Sample | Sample | Sample | Mean | (Log | |
| | 1 (cfu) | 2 (cfu) | 3 (cfu) | (cfu) | cfu) | |
| Control | — | — | — | — | — | |
| 0 h | | | | | | |
| Control | 3.00 × 10⁶ | 2.70 × 10⁷ | 2.30 × 10⁷ | 1.77 × 10⁷ | 7.25 | — |
| 18 h | | | | | | |
| Test | 1.10 × 10⁶ | 1.10 × 10⁶ | 2.30 × 10⁶ | 1.50 × 10⁶ | 6.18 | S = |
| 18 h | | | | | | 1.07 |

Thus, it is seen that the articles obtained from yarns comprising ZnS show high antibacterial activity on the Gram-positive and Gram-negative bacteria, even after 30 washes.

### EXAMPLE 4

### Antifungal Test

The evaluation of the antifungal (antimycosic) activity is measured according to standard ASTM E 2149-01 (shake flask test), according to the procedure adapted by the Hygiene and Biotechnology Laboratory of the Hohenstein Institut (Germany) for fungi. 1 g of test product is placed in contact with 70 ml of a solution of mineral salts and 5 ml of a suspension of Aspergillus niger at 1-3×10⁵ cfu/ml in a 250 ml conical flask. The mineral salt solution was prepared beforehand with the following exact composition:

| | | |
|---|---|---|
| NaNO₃ | 2.0 | g |
| KH₂PO₄ | 0.7 | g |
| K₂HPO₄ | 0.3 | g |
| KCl | 0.5 | g |
| MgSO₄•7H₂O | 0.5 | g |
| FeSO₄•7H₂O | 0.01 | g |
| H₂O | 1000 | ml |
| Tween 80 | 0.1 | g |

A conical flask is prepared in a similar manner with 1 g of control sample. The conical flasks are shaken at a rate of 300 shakes per minute at room temperature. The fungi are counted after 0 and 3 days of incubation.

A degree of reduction R is defined in the following manner:

R=100×(B−A)/B
A=cfu per millilitre for the conical flask containing the sample after 3 days of contact.
B=cfu per millilitre for the conical flask before the contact with the sample (time 0)

The results are given in Table 5:

TABLE 5
Aspergillus niger “von Thieghem”: Strain ATCC 6275
(DSM 1957)

| | | Time 3 days | |
|---|---|---|---|
| | Time 0 (cfu/ml) | (cfu/ml) | R |
| Control | >1.00 × 10⁶ | 1.90 × 10⁶ | R = −90% |
| | | | (increase) |
| Test | 8.00 × 10⁵ | 1.00 × 10⁴ | R = 99% |
| | | | (reduction) |

Thus, it is seen that the articles obtained from yarns comprising ZnS show high antifungal activity.

### EXAMPLE 5

### Preparation of Reels of Yarn and Characterization

The yellowing index and the degradation of the polyamide matrix were compared on yarns comprising ZnS and yarns comprising ZnO.

The polyamide 66 (PA66) used is a polyamide not comprising titanium dioxide, with a relative viscosity of 2.5 (measured at a concentration of 1 g/100 mL in 96% sulphuric acid at 25° C.).

The incorporation of ZnS or ZnO into the PA66 is performed by mixing. The mixture is dried for 20 hours at 100° C. under a vacuum of about 50 mbar and is then introduced into a twin-screw extrusion device which performs the melt-blending. The degree of incorporation of ZnS or ZnO, given in the table below, is calculated relative to the total weight of the composition. The melt is then spun at an adequate die head temperature to produce a yarn (the spinning temperatures are given in the table below) and a speed at the first point of call of 4 200 m/minute, so as to obtain a multi-filament continuous yarn of 42 dtex per 10 filaments. The multifilament or yarn consists of 10 strands (the die consists of 10 holes of 0.38 mm) and the diameter of a strand is about 20 μm.

The yarns obtained were characterized by measuring the molecular mass of the polyamide matrix by GPC (gel permeation chromatography) in dichloromethane after derivatization with trifluoroacetic anhydride, relative to standard polystyrene (PS) solutions. The detection technique used is refractometry. The molecular mass of the matrix is estimated as the maximum of the refractometric peak.

The yarns were also characterized by measuring the yellowing index according to standard YI DIN 6167 (illuminant source: D65).

The results are given in Table 6:

TABLE 6

| | Spinning | Yellowing | GPC (g/mol |
|---|---|---|---|
| Composition | temperature (° C.) | index | equiv. PS) |
| PA 66 control | 283 | 8.7 | 65 000 |
| PA 66 + 0.24% | 283 | 9.4 | 65 000 |
| ZnS | | | |
| PA 66 + 0.5% | 283 | 9.2 | 67 000 |
| ZnS | | | |
| PA 66 + 0.2% | 280 | 13.5 | 56 000 |
| ZnO | | | |
| PA 66 + 0.5% | 271 | 14.8 | 52 000 |
| ZnO | | | |

Thus, ZnS displays much more advantageous capacities in yarns than ZnO in terms of resistance to yellowing and preservation of the polyamide matrix. ZnS is consequently more suitable for introduction into matrices, to obtain yarns, than ZnO, which is known for its antimicrobial properties.

### EXAMPLE 6

### Antifungal Test in Comparison with ZnS Powder

The fungus used is Eurotium amstelodami (strain: CBS 11248). It is cultured in a medium containing 20 g/L of malt extract, 200 g/L of sucrose and 15 g/L of agar. The test samples comprise the following base products:
a polyamide 6 powder with a relative viscosity of 2.6 (measured at 1 g/100 mL in 96% sulphuric acid at 25° C.), ground to a particle size of less than 500 μm;
a masterbatch containing 40% by weight of ZnS in polyamide 6 (reference: Sachtolen PA ZS 40 from Sachtleben, comprising Sachtolith HD-5 from Sachtleben) ground to a particle size of less than 500 μm; and
a ZnS powder (Sachtolith HD-S from Sachtleben).

4 different culture media were manufactured:
medium 1: 20 g/L of malt extract, 200 g/L of sucrose and 15 g/L of agar;
medium 2: medium 1 containing 7.5% by weight of PA 6 powder;
medium 3: medium 1 containing 12.5% by weight of 40% masterbatch powder: i.e. 5% of ZnS equivalent and 7.5% of PA 6 equivalent; and
medium 4: medium 1 containing a powder mixture: 5% by weight of ZnS powder and 7.5% by weight of PA 6 powder (the polyamide not comprising ZnS).

These four media were sterilized and then poured into Petri dishes 85 mm in diameter.

E. amstelodami spores were collected from a 3-week-old culture, suspended in a medium containing 1/1000 of peptone and 1/1000 of Tween 80 and then diluted to obtain 10⁶ spores/ml.

30 μl of suspension were placed in the centre of each test medium. 3 subcultures were prepared for each medium.

The Petri dishes were then incubated at 25° C. under constant white light.

At 12 and 16 days of incubation, the diameter of the colony was measured on each of the media. The results of the averages of the three subcultures are given in the following table:

TABLE 7

| Number of days | Colony diameter (mm) | | | |
|---|---|---|---|---|
| of incubation | Medium 1 | Medium 2 | Medium 3 | Medium 4 |
| 0 | 1 | 1 | 1 | 1 |
| 12 | 78 | 80 | 55 | 61 |
| 16 | 85 | 85 | 60 | 69 |

The variability is plus or minus 1 mm between the different subcultures.

Thus, it is seen that the ZnS contained in the PA 6 causes a strong reduction in the growth of the fungus.

### EXAMPLE 7

### Antibacterial Test in Comparison with ZnS Powder

The test samples comprise the following base products:
a polyamide 6 powder (referred to hereinbelow as powder A) with a relative viscosity of 2.6 (measured at 1 g/100 mL in 96% sulphuric acid, at 25° C.); and
a ZnS powder (Sachtolith HD-S from Sachtleben).

The antibacterial activity is measured according to the same methodology as in Example 2, except that powder is placed in contact with the bacterial suspension.

Control: (A) Extr.:

Powder obtained by extrusion of powder A. The extrusion is performed as follows: the powder is dried for 16 hours at 80° C. under a vacuum of about 50 mbar, and is then introduced into a twin-screw extrusion device. The operating characteristics of the twin-screw extruder are as follows: melt temperature: about 240° C.; melt residence time: 3 minutes. The extrudate obtained at the outlet of the extrusion device is immersed in water at about 20° C. and then crushed and ground, after cooling, with cardice using a Retsch ZM 1000 ultracentrifuge mill. The particle size of the powder obtained is less than 500 μm.

Test 1: (A+ZnS) Extr.:

Powder obtained by mixing 2% by weight of ZnS in powder form with powder A and extrusion of the powder mixture, as mentioned above.

Thus, the powder obtained comprises polyamide 6 granules comprising ZnS.

Test 2: (A) Extr.+ZnS

Powder obtained by mixing 2% by weight of ZnS in powder form with the control powder (A) extr. Thus, the powder obtained comprises polyamide 6 granules and ZnS.

The results are collated in the following table.

TABLE 8
Staphylococcus aureus (Gram-positive): Strain ATCC 6538P

| | | | | | Average | |
|---|---|---|---|---|---|---|
| | Sample | Sample | Sample | Average | (Log | |
| | 1 (cfu) | 2 (cfu) | 3 (cfu) | (cfu) | cfu) | |
| Control | 1.20 × 10⁵ | 1.20 × 10⁵ | 1.50 × 10⁵ | 1.3 × 10⁵ | 5.11 | |
| 0 h | | | | | | |
| Control | 2.90 × 10⁵ | 2.70 × 10⁵ | 3.00 × 10⁵ | 2.87 × 10⁵ | 5.46 | |
| 18 h | | | | | | |
| Test 1 | 1.50 × 10⁴ | 2.50 × 10⁴ | 3.70 × 10⁴ | 2.57 × 10⁴ | 4.41 | S = |
| 18 h | | | | | | 1.05 |
| Test 2 | 3.10 × 10⁵ | 6.30 × 10⁵ | 5.60 × 10⁵ | 5.00 × 10⁵ | 5.70 | S = |
| 18 h | | | | | | −0.24 |

This test shows, surprisingly, that the antibacterial activity of ZnS is obtained when the ZnS is mixed into the polymer matrix.

1.-12. (canceled)

13. Yarns with antibacterial and antifungal properties, comprising a composition comprising at least one polymer matrix and zinc sulphide particles, wherein the zinc sulphide particles do not comprise a mineral coating.

14. The yarns according to claim 13, having a weight proportion of zinc sulphide relative to the total weight of the composition intended to form yarns of between 0.01% and 10%.

15. The yarns according to claim 14, wherein the weight proportion of zinc sulphide is between 0.2% and 5%.

16. The yarns according to claim 13, wherein the polymer matrix is a thermoplastic matrix.

17. The yarns according to claim 16, wherein the thermoplastic matrix comprises at least one thermoplastic polymer selected from the group consisting of polyamides, polyesters, polyolefins, PVC, copolymers and blends thereof.

18. The yarns according to claim 17, wherein the thermoplastic polymer is PET, PBT, PTT, polypropylene or polyethylene.

19. The yarns according to claim 17, wherein the polyamide is polyamide 6, polyamide 6,6, polyamide 11, polyamide 12, polyamide 4, polyamides 4-6, 6-10, 6-12, 6-36 or 12-12; copolymers and blends thereof.

20. The yarns according to claim 13, wherein the zinc sulphide is in the form of particles having a diameter of less than or equal to 5 μm.

21. A composite article with antibacterial and antifungal properties, comprising yarns as defined in claim 13.

22. A process for manufacturing yarns with antibacterial and antifungal properties, comprising the step of spinning a composition comprising a polymer matrix and zinc sulphide.

23. The process according to claim 22, comprising the following steps: a) placing the polymer matrix, optionally in melt form, in contact with zinc sulphide and/or a concentrated composition based on polymer matrix comprising zinc sulphide; and b) spinning the mixture obtained in step a) so as to obtain yarns.

24. A process for providing yarns or articles with antibacterial and antifungal properties comprising the step of adding an efficient antifungal amount of zinc sulphide in a polymer matrix for the manufacture thereof.

25. An article of manufacture with antibacterial and antifungal properties, comprising at least one polymer matrix and zinc sulphide.
